(19) United States
(12) Patent Application Publication
(10) Pub. No.: US 2022/0218511 A1
(43) Pub. Date: Jul. 14, 2022

(54) IMPLANTABLE SURGICAL DEVICE, SYSTEM AND METHOD OF USE

(71) Applicant: AAKASH HASMUKH GAJJAR, HOUSTON, TX (US)

(72) Inventor: AAKASH HASMUKH GAJJAR, HOUSTON, TX (US)

(21) Appl. No.: 17/607,890

(22) PCT Filed: May 1, 2020
(86) PCT No.: PCT/US20/31017

§ 371 Date: Oct. 31, 2021

Related U.S. Application Data
(60) Provisional application No. 62/842,176, filed on May 2, 2019.

(51) Int. Cl. A61F 5/445; A61B 17/34; A61F 5/44

(52) U.S. Cl. CPC A61F 5/445; A61F 2005/4455; A61F 5/4405; A61B 17/3468

(57) ABSTRACT

An implantable surgical device, comprising a body having an inner side separated from an outer side by a body thickness; the body comprising a rigid or semi-rigid portion having an opening disposed therethrough, bound by a perimeter, the body opening dimensioned to allow a portion of a patients bowel to extend therethrough; a plurality of flexible obturators positioned about the opening perimeter, each obturator engaged with, and extending away from the opening perimeter into the body opening, each obturator having an inner face separated from an outer face by a thickness; a portion of at least one obturator overlapping with a portion of an adjacent obturator located on either side of the at least one obturator. A system and method to use the device is also disclosed.

### CROSS REFERENCE TO RELATED APPLICATIONS

This application claims the benefit of and priority to provisional patent application U.S. 62/842,176, filed May 2, 2019, the contents of which are fully incorporated by reference herein.

### BACKGROUND

Various surgical procedures require the forming of an opening in an anatomical wall (i.e., a “stoma” or an “ostomy”), to allow passage of a structure therethrough. When creating an ostomy (e.g., colostomy, ileostomy, and urostomy), a portion of the bowel is relocated through this created opening in the abdominal wall. Depending on the surgical procedure, the bowel is divided, and the free end rerouted through the opening formed in the abdominal wall. The contents of the bowel will empty into a bag or other suitable collection appliance attached to the skin of the patient for the collection and disposal of bowel wastes and/or urine.

The surgical formation of a stoma creates a potential for weakness in the abdominal wall proximate/proximally to the opening. Over time, stretching of the abdominal wall due to normal activity may further weaken the abdominal wall surrounding the stoma, potentially allowing intra-abdominal contents to protrude into or next to the stoma. The end result is a bulge at or near the stoma, referred to as a parastomal hernia.

Numerous intervention strategies have been attempted to prevent the formation of, and/or repair parastomal hernias. These include various forms of surgical mesh which is attached to the abdominal wall about the stoma. See for example US20080167729, US2009029938, US2010037677, US20130030339, and US20140350579, the disclosures of which are fully incorporated by reference herein. However, surgical mesh may result in other unintended consequences.

Other intervention strategies include implantable surgical devices attached to the abdominal wall having an opening coaxial with the stoma through which the bowel is passed through. Such interventions have not been shown to be effective due to herniation between the opening and the bowel. Other strategies have employed a series of flaps disposed about a hole through an implantable device which were arranged to engage the bowel and prevent herniation. See for example US 20050043716 and US20140243588, the disclosures of which are fully incorporated by reference herein. However, this approach has not been shown effective due to a wide variety of issues.

There is a need in the art for an implantable prosthesis or other medical device for the repair and prevention of a hernia around an opening or a stoma.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one or more embodiments of this disclosure, an implantable surgical device comprises a body having an inner side separated from an outer side; the body comprising a semi-rigid to rigid portion having an opening disposed therethrough, bound by a perimeter, the body opening dimensioned to allow a portion of a patients bowel to extend therethrough; a plurality of flexible obturators positioned about the opening perimeter, each obturator engaged with, and extending away from the opening perimeter into the body opening, each obturator having an inner face separated from an outer face by a thickness; a portion of at least one obturator overlapping with a portion of an adjacent obturator located on either side of the at least one obturator.

In one or more embodiments of this disclosure, a system for repairing or resisting the formation of a hernia at or near a stoma formed in an abdominal wall of a patient in which a portion of bowel extends through the stoma, comprises an implantable surgical device comprising a body having an inner side separated from an outer side; the body comprising a rigid or semi-rigid portion having an opening disposed therethrough, bound by a perimeter, the body opening dimensioned to allow a portion of a patients bowel to extend therethrough; a plurality of flexible obturators positioned about the opening perimeter, each obturator engaged with, and extending away from the opening perimeter into the body opening, each obturator having an inner face separated from an outer face by a thickness; a portion of at least one obturator overlapping with a portion of an adjacent obturator located on either side of the at least one obturator; and a cannula, also referred to herein as an introducer, which separate from the implantable surgical device. The introducer having a hollow tubular portion with an outer size (e.g., outer diameter) dimensioned to fit within the body opening to cause the plurality of obturators to deform around the cannula, and an inner size (e.g., an inner diameter) dimensioned to pass the portion of the bowel therethrough, the introducer or cannula being insertable into the body opening and removable from the body opening of said implantable surgical device.

In one or more embodiments of this disclosure, a method of repairing, or reducing the incidence of formation of, a hernia at or near a stoma formed in an abdominal wall for externalizing a portion of bowel, the method comprises the steps of providing an implantable surgical device comprising a body having an inner side separated from an outer side; the body comprising a rigid or semi-rigid portion having an opening disposed therethrough, bound by a perimeter, the body opening dimensioned to allow a portion of a patients bowel to extend therethrough; a plurality of flexible obturators positioned about the opening perimeter, each obturator engaged with, and extending away from the opening perimeter into the body opening, each obturator having an inner face separated from an outer face by a thickness; a portion of at least one obturator overlapping with a portion of an adjacent obturator located on either side of the at least one obturator; providing a cannula or introducer having an outer dimension that is sized to fit within the opening of said implantable surgical device and an inner dimension that is sized to pass the bowel portion therethrough; inserting the cannula into and through the stoma; positioning at least one of the cannula and the implantable surgical device so that an end of the cannula extends through the body opening in the implantable surgical device causing the obturators to flex to accommodate the cannula; inserting the bowel portion into and through the cannula to bring a portion of the bowel external to the patients body; and removing the cannula.

### BRIEF DESCRIPTION OF DRAWINGS

Embodiments of the implantable surgical device, system and method are described with reference to the following figures. The same numbers are used throughout the figures to reference like features and components in which:

FIG. 1 is a top perspective view of an inner face of an implantable surgical device according to one or more embodiments of the invention;

FIG. 2 is a top perspective view of an outer face of an implantable surgical device according to one or more embodiments of the invention;

FIG. 3 is a partial side cut-away view of a body opening of an implantable surgical device according to one or more embodiments of the invention;

FIG. 4 is a top perspective partial view of an inner face of an implantable surgical device showing obturator overlap according to one or more embodiments of the invention;

FIG. 5 is a top perspective partial view of a face of an implantable surgical device according to one or more embodiments of the invention;

FIG. 6 is a top perspective partial view of a face of an implantable surgical device according to one or more alternative embodiments of the invention;

FIG. 7 is a side view of an obturator according to one or more alternative embodiments of the invention;

FIG. 8 is a top perspective view of a face of an implantable surgical device according to one or more embodiments of the invention;

FIG. 9 is a side cut-away view of an implantable surgical device according to one or more embodiments of the invention implanted within a patients abdominal cavity;

FIG. 10a is a side cut-away view of a portion of an implantable surgical device according to one or more embodiments of the invention showing a movable obturator assembly;

FIG. 10b is a side cut-away view of a portion of an implantable surgical device according to one or more embodiments of the invention showing a movable obturator assembly with the obturators at an angle to the body;

FIG. 10c is a side cut-away view of a portion of an implantable surgical device according to one or more embodiments of the invention showing a movable obturator assembly with two layers of obturators at an angle to the body;

FIG. 11 is a top perspective view of a portion of an implantable surgical device according to one or more embodiments of the invention showing a movable obturator assembly;

FIG. 12 is a side cut-away view of an implantable surgical device system according to one or more embodiments of the invention implanted within a patients abdominal cavity utilizing a cannula for placement;

FIG. 13 is a top partial view of a face of an implantable surgical device showing opposite curving obturators according to one or more embodiments of the invention;

FIG. 14 is a side cut-away view of an implantable surgical device system according to one or more embodiments of the invention implanted within a patients abdominal cavity utilizing a cannula and a dilator for placement;

FIG. 15A is a perspective exploded view of an implantable surgical device according to one or more embodiments of the invention showing obturators formed from different layers which are attached to faces and sandwiched between layers of the body; and

FIG. 15B shows a side exploded view according to FIG. 15A.

### DETAILED DESCRIPTION

At the outset, it should be noted that in the development of any such actual embodiment, numerous implementation-specific decisions must be made to achieve the developer's specific goals, such as compliance with system related and business-related constraints, which will vary from one implementation to another. Moreover, it will be appreciated that such a development effort might be complex and time consuming but would nevertheless be a routine undertaking for those of ordinary skill in the art having the benefit of this disclosure. In addition, the device or composition used/disclosed herein can also comprise some components other than those cited. In the summary and this detailed description, each numerical value should be read once as modified by the term “about” (unless already expressly so modified), and then read again as not so modified unless otherwise indicated in context. Also, in the summary and this detailed description, it should be understood that a physical range listed or described as being useful, suitable, or the like, is intended that any and every value within the range, including the end points, is to be considered as having been stated. For example, “a range of from 1 to 10” is to be read as indicating each and every possible number along the continuum between about 1 and about 10. Thus, even if specific data points within the range, or even no data points within the range, are explicitly identified or refer to only a few specific, it is to be understood that inventors appreciate and understand that any and all data points within the range are to be considered to have been specified, and that inventors possessed knowledge of the entire range and all points within the range.

The following definitions are provided in order to aid those skilled in the art in understanding the detailed description.

As used in the specification and claims, “near” is inclusive of “at.” A “polymer” has two or more of the same or different mer units. All numerical values within the detailed description and the claims herein are modified by “about” or “approximately” the indicated value, and take into account experimental error and variations that would be expected by a person having ordinary skill in the art. The term “and/or” refers to both the inclusive “and” case and the exclusive “or” case, whereas the term “and or” refers to the inclusive “and” case only, and such terms are used herein for brevity. For example, a composition comprising “A and/or B” may comprise A alone, B alone, or both A and B; and a composition comprising “A and or B” may comprise A alone, or both A and B.

The term “consisting essentially of” in reference to a composition is understood to mean that in addition to the indicated component(s), the device or composition may further include additional compounds in such amounts and to the extent that the additional compounds do not substantially interfere with the essential function or indicated properties of the overall composition. If no essential function or property is indicated, a composition which consists essentially of an indicated component or composition may include any amount up to 5 percent by weight of the additional components, if any at all.

For purposes herein, ASTM refers to the American Society for Testing and Materials; it is to be understood that when an ASTM method is referred to for use in characterizing a property of a material, the ASTM method referred to is the current revision of the ASTM method in force at the time of filing of this application, unless otherwise indicated.

For purposes herein, “externalization” refers to the relocation of an anatomical structure, such as the bowel, or a prosthetic device or other structure, from the interior side of an anatomical wall to the exterior of the anatomical wall. As used herein, “stoma” refers to an opening in an anatomical wall or other tissue that is naturally or non-naturally formed (e.g., the result of a surgical procedure).

For purposes herein, a tissue-infiltratable surface has a structure and/or a morphology consistent with becoming enmeshed with growing tissue. In other words, a tissue-infiltratable surface has a roughness or coefficient of friction to which growing tissue may attach and/or which allows cellular infiltration. A tissue-infiltratable surface may also comprise holes, a grid structure, or be porous, allowing attachment and/or infiltration of growing tissue. In contrast, a smooth texture which inhibits tissue infiltration is defined herein as a surface which generally resists attachment of growing tissue, at least for a reasonable time, and/or to which tissue may attach, but only weakly as is understood by one of minimal skill in the art. Unless otherwise specified, a smooth surface has a micropore diameter of less than 5 micrometers and/or which has been coated or otherwise treated to prevent cellular infiltration.

For purposes herein, a surgically implantable material is one not known to present toxicity issues, or generally known to cause complications when embedded in the human body. In other words, a surgically implantable material is one generally considered or regarded as safe for implantation by one of skill in the art at the time of filing of this application.

As is commonly understood in the art, an iris valve or iris diaphragm includes a plurality of obturators arranged in an iris pattern. Examples of obturators arranged in an overlapping iris pattern present in the human eye and/or similar to that of a camera lens; examples also include U.S. Pat. Nos. 3,058,483, 7,021,604, and the like.

The implantable surgical device, also referred to herein as a prosthesis and the associated systems and methods of use are described principally in connection with the repair, or resistance to formation of a parastomal hernia associated with an ileostomy, colostomy, urostomy, and other procedures involving externalization of a section of the bowel through a stoma in an abdominal wall. However, it is to be understood that the instant disclosure is not so limited, and the inventive prosthetic device, and the inventive technique of relocating a body structure through a prosthetic device, preferably to limit or avoid contact therebetween so as to reduce the occurrence of potential contamination of the implant, has other applications as should be apparent to one of skill in the art.

In embodiments, an implantable surgical device comprises a body having an inner side separated from an outer side by a body thickness. The body comprising a rigid or semi-rigid portion having an opening disposed therethrough, the opening being bound by a perimeter. The body opening is dimensioned in terms of area and diameter, to allow a portion of a patients bowel to extend therethrough. The device further includes a plurality of flexible obturators positioned about the perimeter of the opening. Each of the obturators is engaged with the body, and extends away from the perimeter of the opening into the body opening. Each of the obturators has an inner face or side separated from an outer face or side, by a thickness. In embodiments, a portion of at least one obturator is positioned to overlap with a portion of another obturator. In embodiments, a portion of at least one obturator is positioned to overlap with a portion of an adjacent obturator located between and/or on either side of the subject obturator about the perimeter of the opening.

In embodiments, greater than 5% of the area of the inner face of an obturator overlaps with the outer face of another obturator. In embodiments, the obturators are arranged about the body opening such that greater than 5% of an area of the outer face of the same obturator overlaps e.g., is in overlapping physical contact with, the inner face of another adjacent obturator. In embodiments, greater than 5% of an area of the inner face of the at least one obturator overlaps with the outer face of one or more non-adjacent obturators, e.g., an obturator which is not directly preceding or directly following the subject obturator about the perimeter of the opening. For example, a non-adjacent obturator may be located across the body opening from the subject obturator, on an opposite side or face of the body, and the like. Likewise, in embodiments, greater than 5% of an area of the outer face of the at least one obturator overlaps with the inner face of one or more non-adjacent obturators. The flexible obturators may be dimensioned and arranged about the body opening, on more than one side of the body, and/or as a plurality of layers such that any combination of overlap is present.

In embodiments, the obturators are arranged radially about the opening such that the inner face of each obturator overlaps with an outer face of an adjacent obturator, and the outer face of each obturator overlaps with an inner face of another adjacent obturator. In embodiments, an edge of a portion of at least one obturator located within the body opening comprises a curved shape. In one or more embodiments, an edge of one or more obturators have a positive curvature with respect to a center point of the body opening. In one or more embodiments, an edge of one or more obturators have a negative curvature with respect to a center point of the body opening. In one or more embodiments, an edge of a first group of obturators have a positive curvature with respect to a center point of the body opening and an edge of a second group of obturators have a negative curvature with respect to a center point of the body opening, wherein one or more of the first group of obturators is in overlapping contact with one or more of the second group of obturators.

In embodiments, at least one obturator comprises a first portion engaged with the body and extending from the opening perimeter into the body opening, and a free end engaged with the body through the first portion, wherein at least a portion of the free end comprises a curved portion extending from the body opening toward the opening perimeter. In embodiments, an edge or side of each obturator has a length greater than a distance between a center point of the body opening and the perimeter. In embodiments, none of the plurality of obturators cover the center point of the body opening. In embodiments, each of the plurality of obturators are overlappingly arranged about the perimeter of the body opening to form an iris-valve arrangement, also referred to as an iris diaphragm arrangement at least partially covering the body opening wherein the obturators are in overlapping contact with one another.

In embodiments, at least a portion of one of the flexible obturators has a hardness greater than about 10 on the Shore 000 scale. In embodiments, preferably one or more of the flexible obturators has a hardness less than or equal to about 80 on the Shore 00 scale, when determined according to ASTM D-2240. In embodiments, the rigid or semi-rigid portion of the body has a hardness greater than or equal to about 10 on the Shore A scale, preferably greater than 10 on the Shore D scale, when determined according to ASTM D-2240. In embodiments, the entire body is rigid or semi-rigid. In embodiments, the obturators have a thickness between sides or faces, from about 0.1 mm to 5 mm.

In embodiments, the inner face of the obturator, the outer face of the obturator, or both, have a smooth texture which inhibits tissue infiltration. In embodiments, an edge of each of the plurality of obturators is engaged with an outer ring forming an obturator assembly, wherein the outer ring of the obturator assembly is rotatably engaged with the body about the body opening such that the obturator assembly including the obturators rotatable about the body opening. In one or more embodiments, a group of obturators are engaged with the first side or face of the body and/or a group of obturators are engaged with the second side or face of the body located opposite the first face and/or a group of obturators are engaged between two or more layers of the body between the first and second sides or faces of the body. In embodiments, an edge of each of the plurality of obturators is engaged with an outer ring forming an obturator assembly which is non-movably engaged with the body about the body opening such that the obturator assembly including the obturators do not rotate about the body opening.

In embodiments, at least a portion of the body comprises a plurality of layers, e.g., is a laminate, wherein the obturators are engaged between at least two of the body layers. In embodiments, the obturators comprise a plurality of layers comprising one or more surgically implantable materials. In embodiments, at least a portion of a first obturator is formed from a first of a plurality of layers of one or more surgically implantable materials, and at least a portion of a second obturator is formed from a second of the plurality of layers of the one or more surgically implantable materials.

In embodiments, at least a portion of the body forms a rigid structure. In some embodiments, at least a portion of the body forms a semi-rigid structure meaning the structure if flexible to allow some movement or bending consistent with the intended use. In some embodiments, at least a portion of the inner side of the body comprises a textured tissue-infiltratable surface. In embodiments, at least a portion of the outer side of the body comprises a smooth surface which prevents tissue infiltration.

In one or more embodiments, the body portion of the device is essentially planer. In other embodiments, at least a portion of the body portion has a curvature or shape corresponding to a curvature or shape of a corresponding internal surface of the patient to which the implantable surgical device is to be affixed. In embodiments, at least a portion of the body is shaped according to previously measured dimensions of a corresponding portion of the abdominal wall of the patient. In embodiments, the body further comprises a plurality of attachment points engaged with and/or disposed therethrough, dimensioned and arranged for attaching the device to an abdominal wall of a patient wherein the body opening is disposed about a surgically created stoma. In embodiments, the attachment points include sutures, holes for sutures, tacks, absorbable tacks, fixation straps, and/or the like, and/or combinations thereof.

In embodiments, each of the plurality of obturators overlap with at least one other obturator, and the obturators have a hardness and are radially arranged about the body opening to flexibly engage a portion of the patients bowel disposed through the body opening without inhibiting peristalsis. In embodiments of such implantable surgical devices, the body has a rigidity which prevents expansion of the stoma, and the obturators have a hardness or flexibility sufficient to prevent herniation, and wherein the obturators have a flexibility such that they slidingly engage with the portion of bowel disposed through the opening.

Embodiments of the invention further include a system for repairing or resisting the formation of a hernia at or near a stoma formed in an abdominal wall of a patient in which a portion of bowel extends through the stoma, comprising an implantable surgical device according to any one or combination of implantable surgical devices according to the instant disclosure, and a tubular introducer or cannula, separate from the implantable surgical device having an outer size dimensioned to fit within the body opening to cause the plurality of obturators to deform around the cannula, and an inner size dimensioned to pass the portion of the bowel therethrough, the cannula being removable from the body opening of said implantable surgical device. In embodiments, the system further includes a dilator, comprising a hollow portion having an inner diameter sized to allow the introducer to pass therethrough. The dilator further having a frustoconical outer shape with a narrow end dimensioned to fit within the body opening, such that as the dilator is introduced through the body opening, the obturators are caused to flex away from the center of the body opening. The introducer may then be inserted through the dilator and the bowel disposed through the introducer. The introducer and the dilator may then be removed allowing the obturators to engage the bowel.

In embodiments, the system further comprises at least one stoma making apparatus sized to fit within the cannula having an end adapted to create a stoma through an anatomical wall of the patient. In embodiments of the system, the cannula further comprises a linear scale for determining the distance from an inner side of the abdominal wall to the epidermis.

Embodiments of the invention further include a method of repairing, or reducing the incidence of formation of, a hernia at or near a stoma formed in an abdominal wall for externalizing a portion of bowel, the method comprising the steps of providing an implantable surgical device according to any one or combination of implantable surgical devices according to the instant disclosure; providing a cannula having an outer dimension that is sized to fit within the opening of said implantable surgical device and an inner dimension that is sized to pass the bowel portion therethrough; inserting the cannula into and through the stoma; positioning at least one of the cannula and the implantable surgical device so that an end of the cannula extends through the opening in the implantable surgical device; inserting the bowel portion into the cannula; and removing the cannula. In embodiments the method further comprises attaching the body of the implantable surgical device to the abdominal wall of the patient. In embodiments the method may further include the steps of providing a stoma forming instrument having an outer dimension that is sized to fit within the cannula; and inserting said stoma forming instrument with the cannula into the abdominal wall. In embodiments the method further comprises the step of positioning the implantable surgical device rearward of the end of the cannula that has been passed through the stoma and into the abdominal cavity. In embodiments, the implantable surgical device is spaced from the cavity entering end of the cannula by at least 1 cm, and/or the cannula is extended through the opening before said step of inserting said bowel portion into the cannula.

In embodiments, the method further comprises the step of sizing the bowel section with a sizing cylinder to determine an appropriately sized stoma forming instrument for forming the stoma. In embodiments, the method includes at least one of a colostomy, ileostomy and an urostomy.

In embodiments, at least a portion of the implantable surgical device provided is dimensioned and contoured to fit within the abdominal cavity of the patient based on previously obtained measurements. In embodiments, at least a portion of the body of the implantable surgical device is produced by three-dimensional printing based on the previously obtained measurements. In such embodiments, the three-dimensional printing includes providing a tissue infiltratable surface on a side of the body to be put into contact with the abdominal wall.

In embodiments, at least a portion of the body of the implantable surgical device comprises an uncured polymeric material, the method further comprises attaching the implantable surgical device to the abdominal wall of the patient, followed by curing the polymeric material to increase the rigidity of the body portion, such that at least a portion of the body is dimensioned and contoured to fit within the abdominal cavity of the patient.

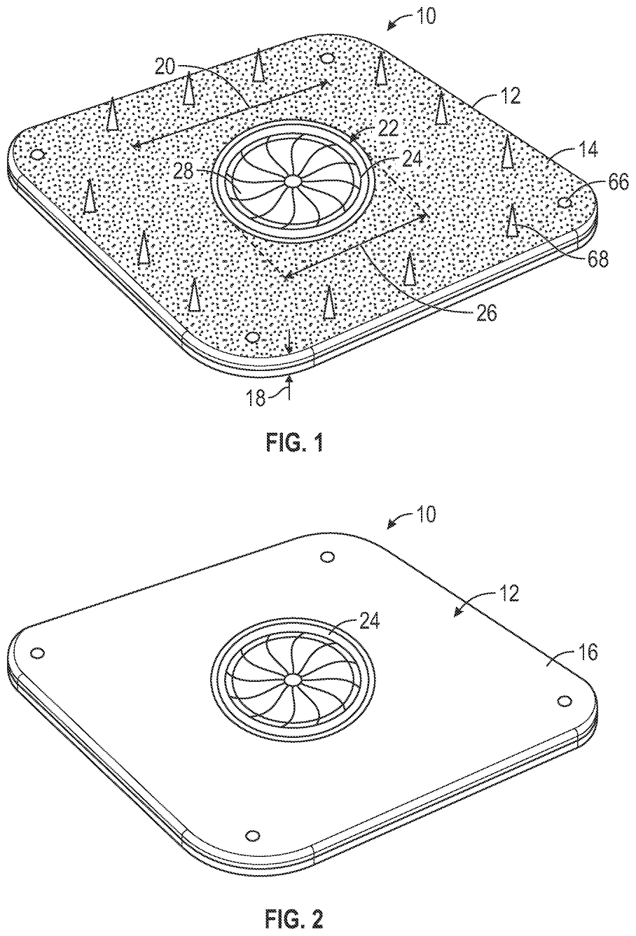

Turning to FIG. 1, an implantable surgical device, generally referred to as 10 for repairing, or resisting the formation of a hernia, particularly a parastomal hernia, is illustrated. The implantable surgical device 10 comprises a body 12 having an inner side or face 14, which is to be oriented in contact with an abdominal wall. The inner face is separated from an outer side or face 16 (see FIG. 2) by a body thickness 18. The outer side 16 being oriented on the side opposite the abdominal wall towards the abdominal cavity. In embodiments, the body thickness 18 is uniform throughout the body. In alternative embodiments, the body thickness 18 varies from point to point. In some embodiments, the body is essentially of solid planer construction, with a body opening disposed through it. In other embodiments, the body is formed in a hub and spoke arrangement from a plurality of spokes disposed about the body opening and extending to an outer frame. Any combination of planer surfaces and/or hub and spoke, and/or other arrangements is suitable for use herein.

In some embodiments, the body 12 comprises a rigid or semi-rigid portion, generally indicated as 20, having an opening 22 disposed therethrough, wherein the body opening 22 is bound by a perimeter 24. In embodiments, the perimeter and/or other surfaces of the body form a rigid or semi-rigid surface having a hardness greater than or equal to about 20 on the Shore A scale, preferably greater than or equal to about 50 on the Shore A scale, or greater than or equal to about 20 on the Shore D scale, or greater than or equal to about 50 on the Shore D scale, when determined according to ASTM D-2240 or an equivalent thereof. The hardness and/or rigidity of the body, along with the structure of the body, serves to strengthen the portion of the abdominal wall and other tissues to which the instant device is attached, with the body hole disposed essentially coaxial with the stoma. The body opening 22 comprises a plurality of sides and is dimensioned, e.g., has a diameter 26 sized to allow a portion of a patients bowel to extend therethrough. The body opening 22 may comprise any number of sides, and is preferably circular. In embodiments, the body opening is generally circular, however it is to be understood that the body opening is not so limited and need only comprise 3 or more sides. In embodiments, diameter 26 of the body opening, which is defined herein as the shortest distance between two opposing sides which goes through a center point 50 of the body opening, is greater than 20 mm and less than 60 mm. In embodiments, the diameter is greater than 25 mm, or greater than 30 mm, or greater than 35 mm, or greater than 40 mm, or greater than 45 mm, and less than 55 mm, or less than 50 mm.

The body opening 22 is at least partially covered by a plurality of flexible obturators 28, positioned about the opening perimeter extending from the perimeter into the body opening.

FIG. 3 shows a partial side on view of an obturator 28 engaged with the body 12 about the perimeter 24 of the body opening 22. For clarity, only one of the plurality of obturators is indicated. Each obturator 28 is engaged with the body proximate to the perimeter 24 of the body opening 22, and extends away from the perimeter 24 into the body opening 22. As shown in FIG. 3, each of the flexible obturators 28 has an inner face 30 separated from an outer face 32 by a thickness 34.

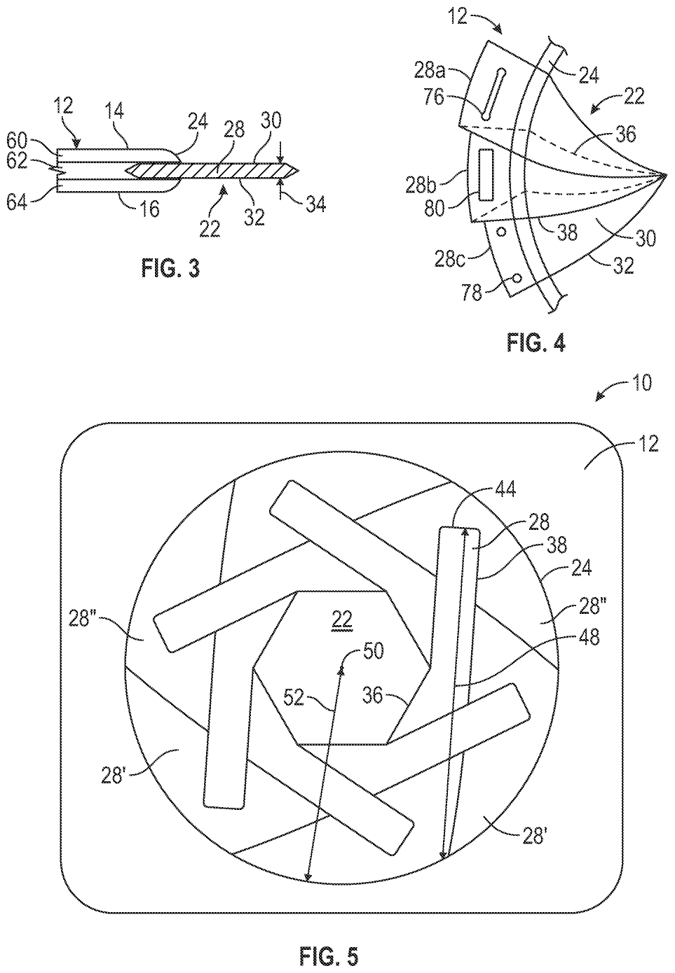

FIG. 4 shows a partial representation of an embodiment in which three (3) of the plurality of obturators 28a, 28b, and 28c are disposed about perimeter 24 and engaged with body 12. The obturators 28 extend away from the perimeter 24 into the body opening 22.

As shown in FIG. 4, in embodiments, a portion of at least one obturator e.g., 28b, extending into the body opening 22 is overlapped by a portion of at least one other adjacent obturator e.g., 28a and/or 28c located on either side or edge 36 and 38 of the subject obturator 28b. In embodiments, greater than 5%, or greater than 10%, or greater than 20%, or greater than 30%, or greater than 50%, of the total area of the outer face 32 of at least one obturator e.g., 28b overlaps with the inner face 30 of the adjacent obturator e.g., 28c within the body opening 22.

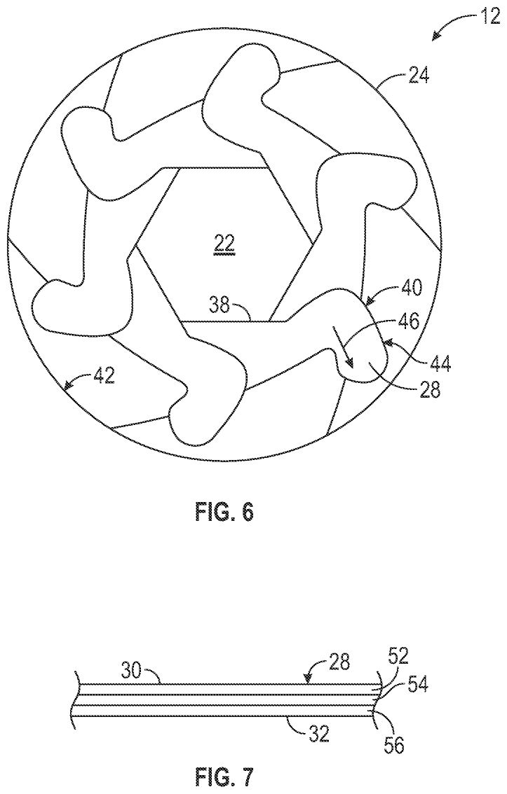

As shown in FIG. 5, in embodiments, a portion of an obturator 28 may also overlap with a portion of a non-adjacent obturator 28″ disposed about the perimeter 24 of the body opening 22. In embodiments, greater than 5%, or greater than 10%, or greater than 20%, of an area of the inner face of the at least one obturator 28 overlaps with the outer face of one or more non-adjacent obturators 28″, and/or greater than 5%, or greater than 10%, or greater than 20%, of an area of the outer face of the at least one obturator 28 overlaps with the inner face of one or more non-adjacent obturators 28″. In embodiments, a side 36 or 38 of each obturator 28 has a length, determined from the perimeter 24 to a free end 44 of the obturator disposed within the body opening 22 which is greater than a distance 52 between a center point 50 of the body opening 22 and the perimeter 24.

In some embodiments, the obturators are dimensioned and arranged such that no portion of any one of the plurality of obturators 28 cover the center point 50 of the body opening 22.

As shown in FIG. 6, in embodiments, an edge 36 and/or 38 of a portion of at least one obturator 28 located within the body opening 22 comprises a curved shape, generally referred to as 40. As is also shown, in embodiments, at least one obturator comprises a first portion 42 engaged with, e.g., anchored to, the body 12 and extending from the opening perimeter 24 into the body opening 22; and a free end 44 only engaged with the body 12 through the first portion 42, wherein at least a portion of the free end 44 comprises a curved portion 40 extending from the body opening 22 toward the opening perimeter 24, as indicated by arrow 46.

In one or more embodiments, the plurality of obturators 28 are overlappingly arranged about the perimeter 24 of the body opening 22 to form an iris valve arrangement, similar to the overlapping arrangement of the iris in the human eye or that seen in a camera, which at least partially covers the body opening. This overlapping arrangement provides for improved resistance to parastomal herniation over devices known in the art. FIG. 13 shows a partial top view of a body opening 22 of an embodiment in which the obturators 28a, 28b, 28c, and 28d have an overlapping arrangement. For clarity, only four obturators are shown. In some embodiments, an outer face of a portion of one obturator may overlap with an inner face of a portion of another obturator and the inner face of a portion of the one obturator overlaps with an outer face of a portion of the other obturator and/or another obturator such that the obturators have a woven arrangement. As shown in FIG. 13, in embodiments the one or more obturators 28a and 28c have a negatively curved portion or edge 40a and 40c with respect to the center point 50 (see FIG. 5), respectively, following an at least partially circular path oriented in a counter-clockwise curvature when determined from the starting point at which the obturator 28 intersects with the perimeter 24. As is also shown in FIG. 13, in embodiments one or more obturators 28b and 28d have a positively curved portion or edge 40b and 40d, respectively, following an at least partially circular path oriented in a clockwise curvature when determined relative to the center point 50 from the starting point at which the obturator 28 intersects with the perimeter 24. As shown in FIG. 13, in embodiments, an obturator having a curvature oriented in a first direction (e.g., a negative or counter clockwise direction) is in overlapping contact with another obturator having a curvature oriented in a second direction (e.g., positive or clockwise direction). In this manner, even though the obturators are flexible and pliable, the overlapping arrangement of the obturators provide support for one another to prevent herniation.

In embodiments, the obturators are flexible, e.g., pliable, supple, easily bent, bendable, pliant, and the like, such that the obturators are capable of bending easily without breaking and are able to be easily modified to respond to altered circumstances or conditions consistent with peristalsis of the bowel without obstructing bowel movement or peristalsis. In embodiments, at least a portion of each obturator has a hardness greater than about 10 on the Shore 000 scale. In embodiments, at least a portion of each obturator has a hardness less than about 80 on the Shore 00 scale. In embodiments, at least a portion of each obturator has a hardness from about 10 on the Shore 000 scale, to about 80 on the Shore 00 scale, when determined according to ASTM D-2240. Preferably the portion of the obturator present within the body opening 22 has a hardness from about 10 to 90 on the Shore 000 scale, from about 10 to 80 on Shore 00 scale, from about 1 to 50 on the shore A scale, or any combination thereof.

Likewise, in embodiments, the obturators have an overall Youngs's modulus of greater than 0.0005 and less than or equal to about 5 GPa, when determined according to ASTM D882, D2136, D790, or an equivalent thereof. In embodiments, the obturators have an overall Youngs's modulus of greater than or equal to about 0.001 GPa, or greater than or equal to about 0.005 GPa, or greater than or equal to about 0.01 GPa, or greater than or equal to about 0.05 GPa, or greater than or equal to about 0.1 GPa, or greater than or equal to about 1 GPa, and less than or equal to about 4 GPa, or less than or equal to about 1 GPa, or less than or equal to about 0.1 GPa, or less than or equal to about 0.01 GPa, when determined according to ASTM D882, D2136, D790, or an equivalent thereof.

In one or more embodiments, at least one of the obturators has a thickness from about 0.1 mm to 5 mm, or from 0.5 to 4 mm, or from 1 to 3 mm thick. In embodiments, the obturators have uniform thickness, on other embodiments, the thickness of the obturator varies along a length of the obturator from a point proximate to the perimeter to the free end disposed within the body opening. In embodiments, the thickness of the obturator decreases along a length of the obturator from a point proximate to the perimeter to the free end disposed within the body opening. In other embodiments, the thickness of the obturator increases along a length of the obturator from a point proximate to the perimeter to the free end disposed within the body opening. In still other embodiments, the thickness of the obturator has an anisotropic variation along a length of the obturator from a point proximate to the perimeter to the free end disposed within the body opening.

In one or more embodiments, the inner face of the obturator 30, the outer face of the obturator 32, or both, have a smooth texture which inhibits tissue infiltration. In one or more embodiments, the one of the inner face of the obturator 30 or the outer face of the obturator 32 has a smooth texture which inhibits tissue infiltration, and the other has a tissue infiltratable surface or texture. In one or more embodiments, the texture of the inner face of the obturator 30 and/or the outer face of the obturator 32 differ between individual obturators.

In embodiments, the obturators are formed from a single material. In alternative embodiments, as shown in FIG. 7, the obturators comprise a laminate, being formed from a plurality of layers, which may be the same or different in composition, physical properties, and/or surface texture. For exemplary purposes, FIG. 7 shows an obturator 28 comprising 3 layers 52, 54, and 56. However, it is to be understood that any one or more of the plurality of obturators may include any number of layers, and that these layers need not be of the same material and/or location from obturator to obturator. In embodiments, the outer layer e.g., 52 and 56 which form the inner face 30 and the outer face 32 of the obturator are formed from one or more surgically implantable materials. Likewise, at least a portion of a second obturator is formed from a second of the plurality of layers of the one or more surgically implantable materials. In embodiments, the inner layer(s) e.g., 54, are formed from a material having improved tensile strength or other physical properties relative to outer layers 52 and/or 56. In embodiments, the layers may include strands, filaments, be a metal foil, and/or the like to provide structural strength, support, and/or resilience for one or more of the more pliable outer layers.

In embodiments, the obturators comprise a plurality of layers comprising one or more surgically implantable materials. In embodiments, the obturators comprise one or more of polytetrafluoroethylene, expanded polytetrafluoroethylene, polyethylene terephthalate, polylactide, polyglycolide, polytrimethylenecarbonate, poly(p-dioxanone), polyurethane, polyethylene homopolymers and/or copolymers, ultrahigh molecular weight polyethylene, polypropylene homopolymers and/or copolymers, hydroxyapatite, stainless steel, titanium and titanium alloys, polysiloxane homopolymers and copolymers (silicones), acrylics, gold, platinum, tantalum, titanium, and other metals and/or alloys thereof from Groups 4 through 12 of the periodic table of the elements, polyether ether ketone, and combinations thereof. Other suitable materials include surgically implantable material and/or tissue products/derivative derived from biologic sources, and the like, including tissues produced using the patient's own cells or tissues.

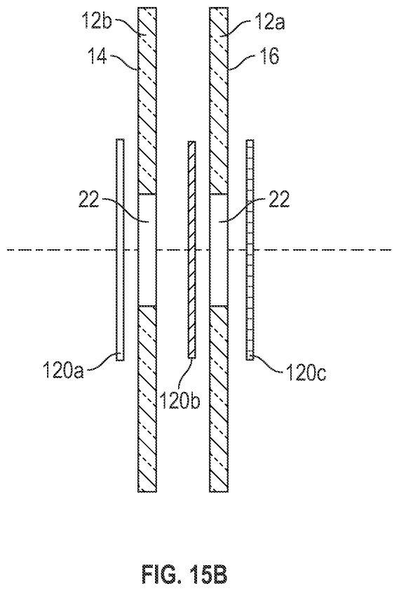

As shown in FIGS. 15A and 15B, in embodiments the obturators 28 are formed from a plurality of layers 120a, 120b and 120c, which may be the same of different, and which have obturators 28 formed therein. For simplicity, only 4 obturators are shown disposed in each layer 120. The orientation of the obturators in the layer, indicated by curved arrows 122a, 122b, and 122c, is offset such that the obturators overlap with one another within the body opening 22. As shown in FIG. 15B, the obturator layers may be disposed on an inner face 14, an outer face 16, or between layers 12a and 12b of the body about the body opening 22.

In embodiments, the body 12 has a rigid or semi-rigid portion. In other embodiments, the entire body is rigid or semi-rigid. In one or more embodiments, the body 12 and/or the rigid/semirigid portion of the body 20 (see FIG. 1) has a hardness greater than or equal to about 50 on the Shore A scale, preferably a hardness greater than or equal to about 10 on the Shore D scale, more preferably greater than or equal to about 50 on the Shore D scale, or greater than or equal to about 70 on the Shore D scale, when determined according to ASTM D-2240.

Returning to FIG. 3, in embodiments, at least a portion of the body 12 comprises a plurality of layers, 60, 62, and 64, e.g., has a laminate structure. In embodiments the obturators 28 are engaged between at least two of the body layers. In other embodiments, the obturators are formed from one or more of the layers of the body laminate 12. As shown in FIG. 4, in embodiments the obturators are attached to the one or more faces 14 and 16 of the body 12 via one or more welds 76, attachment pegs 78, adhesives 80, or a combination thereof, or other forms of securement commonly known in the art.

In one or more embodiments of the implantable surgical device, at least a portion of the body forms a rigid structure. In one or more embodiments at least a portion of the inner side of the body 14 comprises a textured tissue-infiltratable surface. Tissue-infiltratable surfaces may have sufficient roughness, porosity and/or composition which promotes cellular growth thereon and/or therein and/or therethrough. Other suitable surfaces include those comprising scaffolds and other frameworks which support cells to attach, proliferate and differentiate to form an extracellular matrix. Suitable factors include scaffold surface topography and chemical properties, e.g., wettability, softness and stiffness, roughness, microstructure, porosity, pore size, pore shape, interconnectivity, specific surface area and mechanical properties, all of which are known to influence cell behaviors such as adhesion, growth and differentiation, and bioactivity.

In embodiments, either surface of the body and/or the obturator or other components may include an adhesion-forming barrier and/or an antimicrobial substance, such as, for example, metal or antimicrobial metal salt comprising silver, gold, platinum, palladium, iridium, tin, copper, antimony, bismuth, zinc, mercury, lead, cadmium, chromium, thallium, and/or alloys or other compounds, as well as mixtures of any combination of any or all of the foregoing, however, not restricted to the above alone. Examples include, but not limited to, silver chloride, silver nitrate, silver iodide, silver citrate, silver lactate, silver acetate, silver propionate, silver salicylate, silver bromide, silver ascorbate, silver laurel sulfate, silver phosphate, silver sulfate, silver oxide, silver benzoate, silver carbonate, silver sulfadiazine, and silver gluconate, as disclosed in US2005085924, the disclose or which is incorporated by reference herein.

Other suitable antimicrobial substances include, but are not limited to: biguanide substances, such as chlorhexidine and its salts, triclosan, penicillins, tetracyclines, aminoglycosides, such as gentamicin and tobramycin, polymyxins, rifampicins, bacitracins, erythromycins, vancomycins, neomycins, chloramphenicols, miconazole, quinolones, such as oxolinic acid, norfloxacin, nalidixic acid, pefloxacin, enoxacin, and ciprofloxacin, sulfonamides, nonoxynol 9, fusidic acid, cephalosporins, and combinations of such substances and similar substances. Antibiotic ceramic particles useful with the present invention include zeolites, hydroxyapatite, zirconium phosphates or other ion-exchange ceramics. Hydroxyapatite particles containing antimicrobial metals are described, e.g., in U.S. Pat. No. 5,009,898. Zirconium phosphates containing antimicrobial metals are described, e.g., in U.S. Pat. Nos. 5,296,238; 5,441,717; and 5,405,644. Antibiotic zeolites may be prepared by replacing all or part of the ion-exchangeable ions in zeolite with ammonium ions and antibiotic metal ions, as described in U.S. Pat. Nos. 4,938,958 and 4,911,898. Such zeolites may be incorporated in antibiotic resins (see U.S. Pat. Nos. 4,938,955 and 4,906,464) and polymer articles (see U.S. Pat. No. 4,775,585). Polymers including the antibiotic zeolites have been used to make different kinds of articles, see U.S. Pat. Nos. 5,714,445; 5,697,203; 5,562,872; 5,180,585; 5,714,430; and 5,102,401.

Likewise, in embodiments at least a portion of the outer side of the body comprises a smooth surface which prevents tissue infiltration. Typical smooth surfaces which prevents tissue infiltration include those having a microporous surface having an average pore size of less than about 10, preferably less than about 5 micrometers.

In an embodiment, the non-tissue infiltratable portion of the body may comprise or consists essentially of expanded polytetrafluoroethylene (e.g., GORE-TEX available from W. L. Gore & Associates, Inc., having a pore size (submicronal) that discourages tissue ingrowth and adhesion. Other suitable materials include silicone elastomer, e.g., SILASTIC Rx Medical Grade Sheeting (Platinum Cured) distributed by Dow Corning Corporation, TEFLON mesh, microporous polypropylene sheeting (CELGARD), collagen, hyaluronic acid, carboxymethyl cellulose, and glycolic acid polymers. Autogenous, heterogeneous, and xenogeneic tissue also are contemplated including, for example, pericardium and small intestine submucosa, biodegradable and/or absorbable materials, such as oxidized, regenerated cellulose (INTERCEED) may be employed and/or hydrogels or combinations thereof.

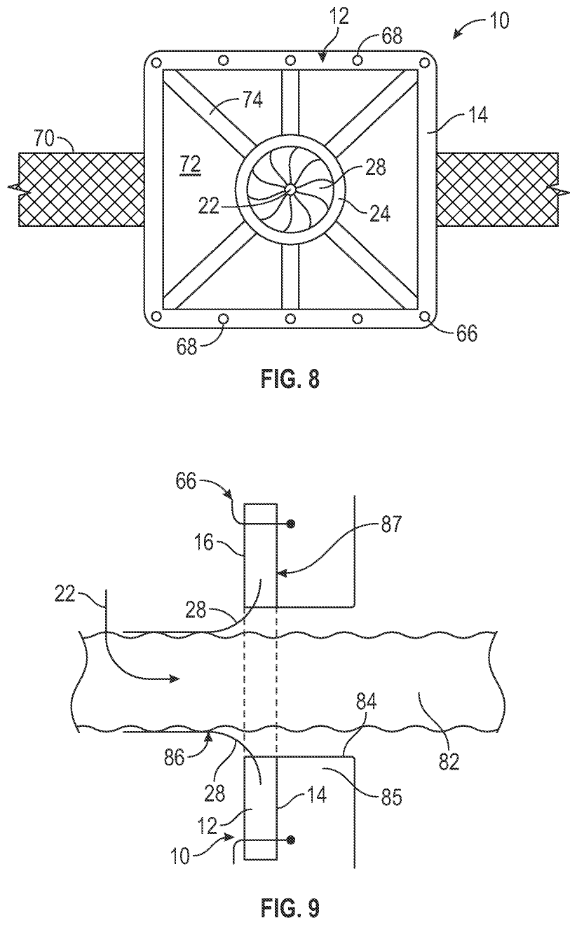

As shown in FIG. 1, in embodiments the implantable surgical device comprises one or more attachment points which may include absorbable/nonabsorbable sutures and/or holes for sutures 66, tacks 68 which may also be absorbable tacks, and the like. As shown in FIG. 8, in one or more embodiments, the implantable surgical device may include fixation straps 70, or any combination of fixation and/or attachment devices.

As shown in FIG. 1, in embodiments the body portion 12 may be essentially monolithic. In other embodiments, as shown in FIG. 8, the body portion 12 may include open spaces 72 at least partially surrounded by one or more rigid and/or flexible frame members 74.

In embodiments, the body is essentially planer. In alternative embodiments, as shown in FIG. 9, at least a portion of the body has a curvature or shape 87 consistent with a curvature of a corresponding internal surface of the patient. In one or more embodiments, the body portion may have a shape dimensioned and arranged to conform to the abdominal wall of the patient according to previously measured dimensions of a corresponding portion of the patient. For example, the contour of a patients abdominal wall as determined using MRI, ultrasound and/or other imaging methods known in the art.

As shown in FIG. 9, in embodiments, each of the plurality of flexible obturators 28 overlap with at least one other flexible obturator 28, and the obturators have a hardness and are radially arranged about the body opening 22 to flexibly engage a portion of the patients bowel 82 disposed through the body opening 22 without inhibiting peristalsis.

In embodiments, the body 12 has a rigidity and the obturators 28 have a hardness sufficient to prevent herniation through the surgically created stoma 84. In embodiments, the obturators 28 have a flexibility such that they slidingly engaged 86 with the portion of bowel 82 disposed through the body opening 22 and the stoma 84 disposed through the abdominal wall 85.

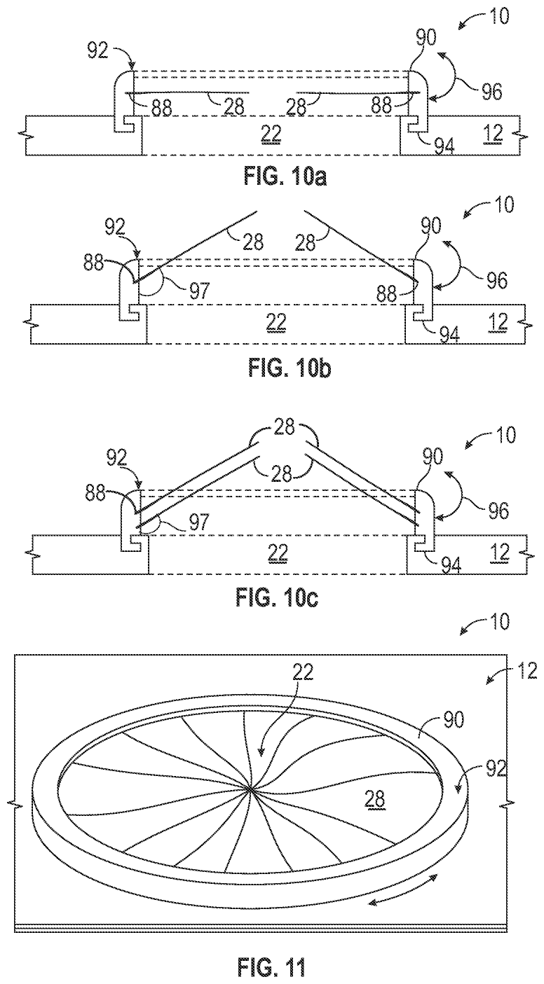

As shown in FIGS. 10a, 10b, and 10c, which are side-on views of an implantable surgical device according to embodiments of the invention, and in FIG. 11 which is a perspective top view, an attachment edge 88 of each of the plurality of obturators 28 is engaged with an outer ring 90 forming an obturator assembly 92. The outer ring 90 of the obturator assembly 92 is rotatably engaged 94 with the body 12 about the body opening 22 such that the obturator assembly 92 may be rotatable 96 about the body opening 22. In other embodiments, the obturator assembly is non-movably engaged with the body. In other embodiments, the obturators are engaged with the outer ring such that rotation of the ring increases or decreases the amount of the body opening occupied by the obturators. Suitable mechanisms for such adjustment include those disclosed in U.S. Pat. Nos. 8,075,482, 7,021,604, 6,440,063, 5,906,577, 3,666,136, 3,058,483, and the like. As is further shown in FIG. 10a, in embodiments the obturators 28 are oriented essentially parallel to the plane of the opening, i.e., parallel to the body 12. In an alternative embodiment shown in FIG. 10b, the obturators 28 are oriented at an angle 97 of less than about 90° relative to the plane of the opening. As shown in FIG. 10c, in some embodiments, two or more rows of obturator 28 may be oriented at different angles, e.g., one set parallel to the body 12 and the other set at an acute angle 97 to the body, and/or, two sets parallel to the body 12 and/or two sets at an acute angle 97 to the body. Accordingly, in embodiments, the plurality of obturators are oriented at an angle relative to a plane of the body opening extending towards the interior of the patient, away from the abdominal wall side of the body. It is to be understood that the obturators may be disposed at an angle to the body independent of the outer ring being rotatably engaged 94 with the body 12 about the body opening 22.

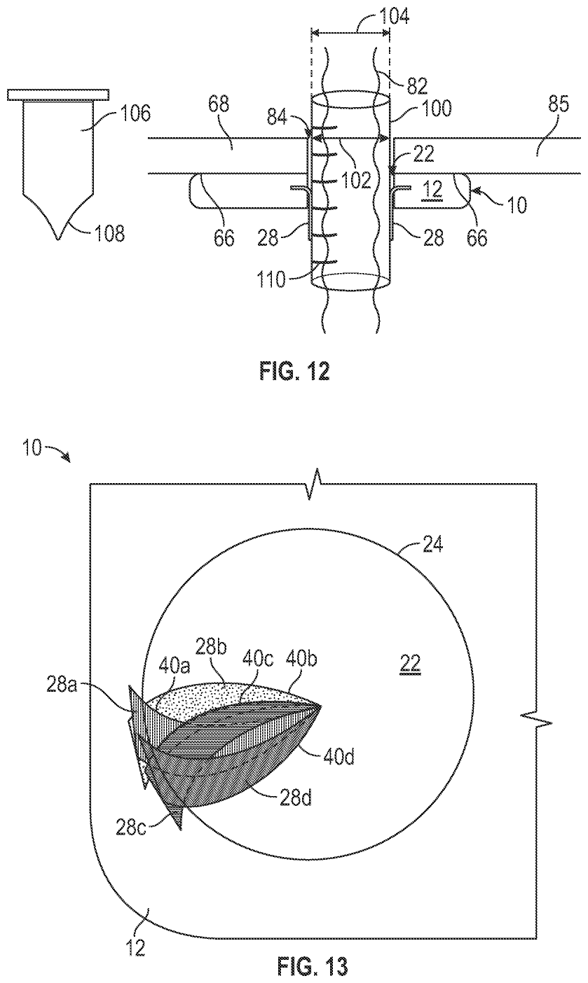

As shown in FIG. 12, in embodiments, a system for repairing or resisting the formation of a hernia at or near a stoma formed in an abdominal wall of a patient in which a portion of bowel extends through the stoma, comprises an implantable surgical device 10 according to any one or combination of embodiments disclosed herein, along with a cannula 100, separate from the implantable surgical device 10, having an outer size 104 dimensioned to fit within the body opening 22 to cause the plurality of obturators 28 to deform around the cannula 100, and an inner size 102 dimensioned to pass the portion of the bowel 82 therethrough, the cannula 100 being removable from the body opening 22 of said implantable surgical device in which the center of the body opening 22 is coaxial with a center of the stoma 84, once the bowel 82 is disposed therethrough.

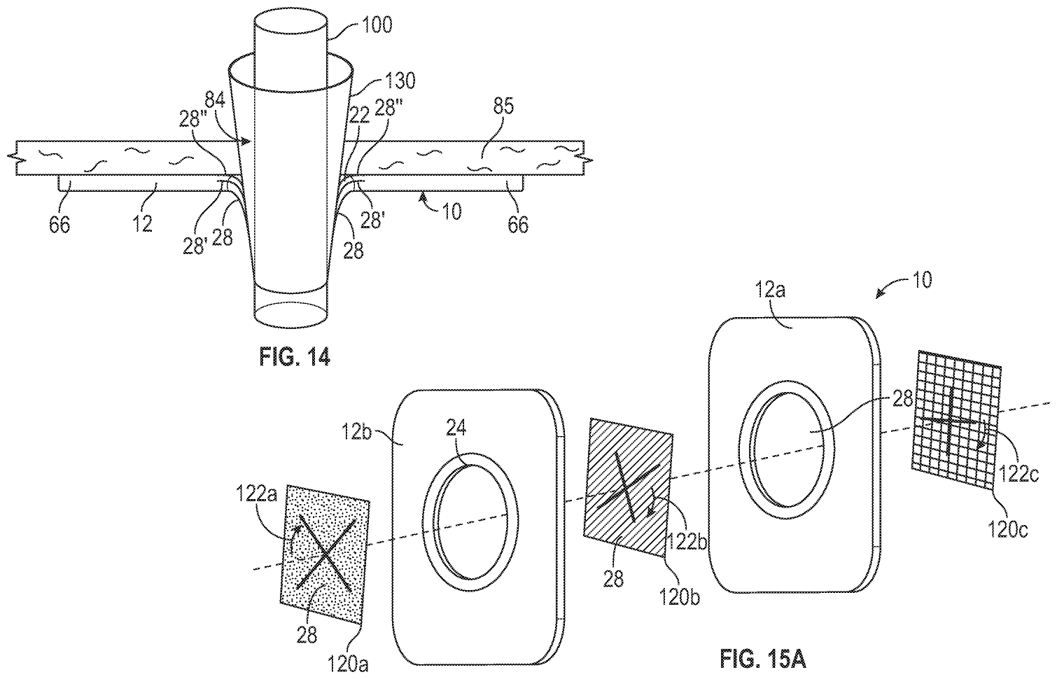

As shown in FIG. 14, the system for repairing or resisting the formation of a hernia at or near a stoma formed in an abdominal wall of a patient in which a portion of bowel extends through the stoma, comprises an implantable surgical device 10 according to any one or combination of embodiments disclosed herein, may further include a dilator 130, having a frustoconical shape dimensioned to fit within the body opening 22 to cause the plurality of obturators 28 to deform, and an inner diameter through which the cannula 100 fits to assist in inserting the cannula 100, or as a replacement for the cannula 100. As shown in the figure, three sets of obturators 28, 28′ and 28″, which are attached to an outer side 16, between the sides 16 and 14, and an inner side 14 of the body 12, respectively.

As is shown in FIG. 12, in one or more embodiments, the system may further comprise at least one stoma making apparatus 106 sized to fit within the cannula 100 having an end 108 adapted to create a stoma 84 through an anatomical wall 85 of the patient.

In embodiments the cannula further comprises a linear scale 110 for determining the distance from an inner side of the abdominal wall to the epidermis.

In one or more embodiments a method of repairing, or reducing the incidence of formation of, a hernia at or near a stoma formed in an abdominal wall for externalizing a portion of bowel comprises the steps of providing an implantable surgical device according to any one or combination of embodiments disclosed herein, providing a cannula having an outer dimension that is sized to fit within the opening of said implantable surgical device and an inner dimension that is sized to pass the bowel portion therethrough; inserting the cannula into and through the stoma; positioning at least one of the cannula and the implantable surgical device so that an end of the cannula extends through the opening in the implantable surgical device; inserting the bowel portion into the cannula; and removing the cannula.

In embodiments, the method further includes attaching the body of the implantable surgical device to the abdominal wall of the patient, and/or

providing a stoma forming instrument having an outer dimension that is sized to fit within the cannula; and inserting said stoma forming instrument with the cannula into the abdominal wall; and/or
positioning the implantable surgical device rearward of the end of the cannula that has been passed through the stoma and into the abdominal cavity; and/or
wherein the implantable surgical device is spaced from the cavity entering end of the cannula by at least 1 cm; and/or
wherein the cannula is extended through the opening before said step of inserting said bowel portion into the cannula; and/or
sizing the bowel section with a sizing cylinder to determine an appropriately sized stoma forming instrument for forming the stoma. In embodiments any of the above steps is included in at least one of a colostomy, ileostomy and an urostomy.

In embodiments, at least a portion of the implantable surgical device is dimensioned and contoured to fit within the abdominal cavity of the patient based on previously obtained measurements. In embodiments, at least a portion of the body of the implantable surgical device is produced by three-dimensional printing based on the previously obtained measurements. In embodiments, the three-dimensional printing includes providing a tissue infiltratable surface on a side of the body to be put into contact with the abdominal wall. In alternative embodiments, the method may include an implantable surgical device in which a portion of the body comprises an uncured polymeric material, the method further comprising attaching the implantable surgical device to the abdominal wall of the patient, followed by curing the polymeric material such that at least a portion of the body is dimensioned and contoured to fit within the abdominal cavity of the patient, as described by US20040091603, and the like, the disclosure of which is incorporated by reference herein.

In embodiments the body opening 22 has a size equal to or slightly smaller than the entry to the stoma formed through the abdominal wall, so that gaps between the body portion and the entrance to the stoma are avoided which might otherwise be vulnerable to herniation.

As is evident from the figures and text presented above, a variety of embodiments are contemplated:
E1. An implantable surgical device, comprising:
   a body having an inner side separated from an outer side by a body thickness;
   the body comprising a rigid or semi-rigid portion having an opening disposed therethrough, bound by a perimeter, the body opening dimensioned to allow a portion of a patients bowel to extend therethrough;
   a plurality of flexible obturators positioned about the opening perimeter, each obturator engaged with, and extending away from the opening perimeter into the body opening, each obturator having an inner face separated from an outer face by a thickness;
   a portion of at least one obturator overlapping with a portion of an adjacent obturator located on either side of the at least one obturator.
E2. The implantable surgical device according to embodiment E1, wherein greater than 5% of an area of the inner face of the at least one obturator overlaps with the outer face of the adjacent obturator;
   wherein greater than 5% of an area of the outer face of the at least one obturator overlaps with the inner face of the adjacent obturator;
   wherein greater than 5% of an area of the inner face of the at least one obturator overlaps with the outer face of one or more non-adjacent obturators;
   wherein greater than 5% of an area of the outer face of the at least one obturator overlaps with the inner face of one or more non-adjacent obturators; or a combination thereof.
E3. The implantable surgical device according to embodiment E1 or E2, wherein the flexible obturators are arranged radially about the opening such that the inner face of each obturator overlaps with an outer face of an adjacent obturator, and the outer face of each obturator overlaps with an inner face of another adjacent obturator.
E4. The implantable surgical device according to any one of embodiments E1 through E3, wherein an edge of a portion of at least one obturator located within the body opening comprises a curved shape.
E5. The implantable surgical device according to any one of embodiments E1 through E4, wherein at least one obturator comprises a first portion engaged with the body and extending from the opening perimeter into the body opening, and a free end engaged with the body through the first portion, wherein at least a portion of the free end comprises a curved portion extending from the body opening toward the opening perimeter.
E6. The implantable surgical device according to any one of embodiments E1 through E5, wherein a side of each obturator has a length greater than a distance between a center point of the body opening and the perimeter.
E7. The implantable surgical device according to any one of embodiments E1 through E6, wherein none of the plurality of obturators cover the center point of the body opening.
E8. The implantable surgical device according to any one of embodiments E1 through E7, wherein the plurality of obturators are overlappingly arranged about the perimeter of the body opening to form an iris valve arrangement at least partially covering the body opening and/or wherein the plurality of obturators are oriented at an angle relative to a plane of the body opening.
E8a. The implantable surgical device according to any one of embodiments E1 through E8, wherein the plurality of obturators are oriented at an angle relative to a plane of the body opening, preferably away from the side of the body to contact the abdominal wall of the patient, and/or two or more layers of the obturators are oriented at one or more angles relative to the plane of the body.
E9. The implantable surgical device according to any one of embodiments E1 through E8a, wherein at least a portion of one of the obturators has a hardness from about 10 on the Shore 000 scale, to about 80 on the Shore 00 scale, when determined according to ASTM D-2240.
E10. The implantable surgical device according to any one of embodiments E1 through E9, wherein at least one of the obturators has a thickness from about 0.1 mm to 5 mm.
E11. The implantable surgical device according to any one of embodiments E1 through E10, wherein the inner face of the obturator, the outer face of the obturator, or both, have a smooth texture which inhibits tissue infiltration.
E12. The implantable surgical device according to any one of embodiments E1 through E11, wherein at least a portion of a first obturator is formed from a first of a plurality of layers of one or more surgically implantable materials, and at least a portion of a second obturator is formed from a second of the plurality of layers of the one or more surgically implantable materials.
E13. The implantable surgical device according to any one of embodiments E1 through E12, wherein the rigid portion of the body has a hardness greater than or equal to about 50 on the Shore A scale, when determined according to ASTM D-2240.
E14. The implantable surgical device according to any one of embodiments E1 through E13, wherein the entire body is rigid or semi-rigid.
E15. The implantable surgical device according to any one of embodiments E1 through E14, wherein at least a portion of the body comprises a single or plurality of layers, and wherein the obturators are engaged between at least two of the body layers.
E16. The implantable surgical device according to any one of embodiments E1 through E15, wherein the obturators comprise a plurality of layers comprising one or more surgically implantable materials.
E17. The implantable surgical device according to any one of embodiments E1 through E16, wherein each of the plurality of flexible obturators overlap with at least one other flexible obturator, and wherein the obturators have a hardness and are radially arranged about the body opening to flexibly engage a portion of the patients bowel disposed through the body opening without inhibiting peristalsis.
E18. The implantable surgical device according to any one of embodiments E1 through E17, wherein the body has a rigidity and the obturators have a hardness sufficient to prevent herniation, and wherein the obturators have a flexibility such that they slidingly engage with the portion of bowel disposed through the opening.
E19. The implantable surgical device according to any one of embodiments E1 through E18, wherein an edge of each of the plurality of obturators is engaged with an outer ring forming an obturator assembly, wherein the outer ring of the obturator assembly rotatably engaged with the body about the body opening such that the obturator assembly is rotatable about the body opening.
E19a. The implantable surgical device according to any one of embodiments E1 through E18, wherein an edge of each of the plurality of obturators is engaged with an outer ring forming an obturator assembly, wherein the outer ring of the obturator assembly is non-movably engaged with the body about the body opening such that the obturator assembly is fixed about the body opening.
E20. The implantable surgical device according to any one of embodiments E1 through E19a, where at least a portion of the body forms a rigid or semi-rigid structure, and wherein at least a portion of the inner side of the body comprises a textured tissue-infiltratable surface.
E21. The implantable surgical device according to any one of embodiments E1 through E20, wherein at least a portion of the outer side of the body comprises a smooth surface which prevents tissue infiltration.
E22. The implantable surgical device according to any one of embodiments E1 through E21, wherein the body is essentially planer.
E23. The implantable surgical device according to any one of embodiments E1 through E22, wherein at least a portion of the body has a curvature consistent with a curvature of a corresponding internal surface of the patient.
E24. The implantable surgical device according to any one of embodiments E1 through E23, wherein at least a portion of the body is shaped according to previously measured dimensions of a corresponding portion of the abdominal wall of the patient.
E25. The implantable surgical device according to any one of embodiments E1 through E24, wherein the body further comprises a plurality of attachment points engaged with and/or disposed therethrough, dimensioned and arranged for attaching the device to an abdominal wall of a patient wherein the body opening is disposed about a surgically created stoma.
E26. The implantable surgical device according to any one of embodiments E1 through E25, wherein the attachment points include sutures, holes for sutures, tacks, absorbable tacks, fixation straps, or a combination thereof
S27. A system for repairing or resisting the formation of a hernia at or near a stoma formed in an abdominal wall of a patient in which a portion of bowel extends through the stoma, comprising:
   an implantable surgical device according to any one of embodiments E1 through E26; and
   a cannula, separate from the implantable surgical device, having an outer size dimensioned to fit within the body opening to cause the plurality of obturators to deform around the cannula, and an inner size dimensioned to pass the portion of the bowel therethrough, the cannula being removable from the body opening of said implantable surgical device.
S28. The system according to embodiment S27, further comprising at least one stoma making apparatus sized to fit within the cannula having an end adapted to create a stoma through an anatomical wall of the patient.
S29. The system according to embodiment S27 or S28, wherein the cannula further comprises a linear scale for determining the distance from an inner side of the abdominal wall to the epidermis.
M30. A method of repairing, or reducing the incidence of formation of, a hernia at or near a stoma formed in an abdominal wall for externalizing a portion of bowel, the method comprising the steps of:
   providing an implantable surgical device according to any one of embodiments E1 through E26;
   providing a cannula having an outer dimension that is sized to fit within the opening of said implantable surgical device and an inner dimension that is sized to pass the bowel portion therethrough;
   inserting the cannula into and through the stoma;
   positioning at least one of the cannula and the implantable surgical device so that an end of the cannula extends through the opening in the implantable surgical device; inserting the bowel portion into the cannula; and removing the cannula.
M31. The method of embodiment M30, further comprising attaching the body of the implantable surgical device to the abdominal wall of the patient.
M32. The method of embodiment M30 or M31, further comprising the steps of:
   providing a stoma forming instrument having an outer dimension that is sized to fit within the cannula; and inserting said stoma forming instrument with the cannula into the abdominal wall.
M33. The method of any one of embodiments M30 through M32, further comprising the step of positioning the implantable surgical device rearward of the end of the cannula that has been passed through the stoma and into the abdominal cavity.
M34. The method of any one of embodiments M30 through M33, wherein the implantable surgical device is spaced from the cavity entering end of the cannula by at least 1 cm.
M35. The method of any one of embodiments M30 through M34, wherein the cannula is extended through the opening before said step of inserting said bowel portion into the cannula.
M36. The method of any one of embodiments M30 through M35, further comprising the step of sizing the bowel section with a sizing cylinder to determine an appropriately sized stoma forming instrument for forming the stoma.
M37. The method of any one of embodiments M30 through M36 including at least one of a colostomy, ileostomy and an urostomy.
M38. The method of any one of embodiments M30 through M37, wherein at least a portion of the implantable surgical device is dimensioned and contoured to fit within the abdominal cavity of the patient based on previously obtained measurements.
M39. The method of any one of embodiments M30 through M38, wherein at least a portion of the body of the implantable surgical device is produced by three-dimensional printing based on the previously obtained measurements.
M40. The method of any one of embodiments M30 through M39, wherein the three-dimensional printing includes providing a tissue infiltratable surface on a side of the body to be put into contact with the abdominal wall.
M41. The method of any one of embodiments M30 through M40, wherein at least a portion of the body of the implantable surgical device comprises an uncured polymeric material, the method further comprising attaching the implantable surgical device to the abdominal wall of the patient, followed by curing the polymeric material such that at least a portion of the body is dimensioned and contoured to fit within the abdominal cavity of the patient.

It should be understood that the foregoing description of the invention is intended merely to be illustrative thereof and that other equivalents, embodiments and modifications of the invention may be apparent to those skilled in the art.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. § 112 for any limitations of any of the claims herein, except for those in which the claim expressly uses the words ‘means for’ together with an associated function.

I claim:

1. An implantable surgical device, comprising:
a body having an inner side separated from an outer side by a body thickness;
the body comprising a rigid or semi-rigid portion having an opening disposed therethrough, bound by a perimeter, the body opening dimensioned to allow a portion of a patient's bowel to extend therethrough;
a plurality of flexible obturators positioned about the opening perimeter, each obturator engaged with, and extending away from the opening perimeter into the body opening, each obturator having an inner face separated from an outer face by a thickness;
a portion of at least one obturator overlapping with a portion of an adjacent obturator located on either side of the at least one obturator.

2. The implantable surgical device of claim 1, wherein the flexible obturators are arranged radially about the opening such that the inner face of each obturator overlaps with an outer face of an adjacent obturator, and the outer face of each obturator overlaps with an inner face of another adjacent obturator.

3. The implantable surgical device of claim 1, wherein an edge of a portion of at least one obturator located within the body opening comprises a curved shape.

4. The implantable surgical device of claim 1, wherein a side of each obturator has a length greater than a distance between a center point of the body opening and the perimeter.

5. The implantable surgical device of claim 1, wherein the plurality of obturators are overlappingly arranged about the perimeter of the body opening to form an iris valve arrangement at least partially covering the body opening.

6. The implantable surgical device of claim 1, wherein the plurality of obturators are oriented at an angle relative to a plane of the body opening.

7. The implantable surgical device of claim 1, wherein at least a portion of one of the obturators has a hardness from about 10 on the Shore 000 scale, to about 80 on the Shore 00 scale, when determined according to ASTM D-2240.

8. The implantable surgical device of claim 1, wherein at least one of the obturators has a thickness from about 0.1 mm to 5 mm.

9. The implantable surgical device of claim 1, wherein the inner face of the obturator, the outer face of the obturator, or both, have a smooth texture which inhibits tissue infiltration.

10. The implantable surgical device of claim 1, wherein at least a portion of a first obturator is formed from a first of a plurality of layers of one or more surgically implantable materials, and at least a portion of a second obturator is formed from a second of the plurality of layers of the one or more surgically implantable materials.

11. The implantable surgical device of claim, wherein the rigid or semi-rigid portion of the body has a hardness greater than or equal to about 50 on the Shore A scale, when determined according to ASTM D-2240.

12. The implantable surgical device of claim, wherein the entire body is rigid.

13. The implantable surgical device of claim 1, wherein at least a portion of the body comprises a plurality of layers, and wherein the obturators are engaged between at least two of the body layers.

14. The implantable surgical device of claim 1, wherein each of the plurality of flexible obturators overlap with at least one other flexible obturator, and wherein the obturators have a hardness and are radially arranged about the body opening to flexibly engage a portion of the patients bowel disposed through the body opening without inhibiting peristalsis, and wherein the body has a rigidity and the obturators have a hardness sufficient to prevent herniation.

15. The implantable surgical device of claim 1, wherein an edge of each of the plurality of obturators is engaged with an outer ring forming an obturator assembly, wherein the outer ring of the obturator assembly rotatably engaged with the body about the body opening such that the obturator assembly is rotatable about the body opening.

16. The implantable surgical device of claim 1, where at least a portion of the body forms a rigid or semi-rigid structure, and wherein at least a portion of the inner side of the body comprises a textured tissue-infiltratable surface and/or wherein at least a portion of the outer side of the body comprises a smooth surface which prevents tissue infiltration.

17. The implantable surgical device of claim 1, wherein at least a portion of the body has a curvature consistent with a curvature of a corresponding internal surface of the patient, and/or wherein at least a portion of the body is shaped according to previously measured dimensions of a corresponding portion of the abdominal wall of the patient.

18. The implantable surgical device of claim 1, wherein the body further comprises a plurality of attachment points engaged with and/or disposed therethrough, dimensioned and arranged for attaching the device to an abdominal wall of a patient wherein the body opening is disposed about a surgically created stoma.

19. A system for repairing or resisting the formation of a hernia at or near a stoma formed in an abdominal wall of a patient in which a portion of bowel extends through the stoma, comprising:
an implantable surgical device comprising a body having an inner side separated from an outer side by a body thickness;
the body comprising a rigid or semi-rigid portion having an opening disposed therethrough, bound by a perimeter, the body opening dimensioned to allow a portion of a patients bowel to extend therethrough;
a plurality of flexible obturators positioned about the opening perimeter, each obturator engaged with, and extending away from the opening perimeter into the body opening, each obturator having an inner face separated from an outer face by a thickness;
a portion of at least one obturator overlapping with a portion of an adjacent obturator located on either side of the at least one obturator; and
a cannula, separate from the implantable surgical device, having an outer size dimensioned to fit within the body opening to cause the plurality of obturators to deform around the cannula, and an inner size dimensioned to pass the portion of the bowel therethrough, the cannula being removable from the body opening of said implantable surgical device, and optionally comprising at least one stoma making apparatus sized to fit within the cannula having an end adapted to create a stoma through an anatomical wall of the patient.

20. A method of repairing, or reducing the incidence of formation of, a hernia at or near a stoma formed in an abdominal wall for externalizing a portion of bowel, the method comprising the steps of:
providing an implantable surgical device comprising a body having an inner side separated from an outer side by a body thickness;
the body comprising a rigid portion having an opening disposed therethrough, bound by a perimeter, the body opening dimensioned to allow a portion of a patient's bowel to extend therethrough;
a plurality of flexible obturators positioned about the opening perimeter, each obturator engaged with, and extending away from the opening perimeter into the body opening, each obturator having an inner face separated from an outer face by a thickness;
a portion of at least one obturator overlapping with a portion of an adjacent obturator located on either side of the at least one obturator;
providing a cannula having an outer dimension that is sized to fit within the opening of said implantable surgical device and an inner dimension that is sized to pass the bowel portion therethrough;
inserting the cannula into and through the stoma;
positioning at least one of the cannula and the implantable surgical device so that an end of the cannula extends through the opening in the implantable surgical device; inserting the bowel portion into the cannula; and
removing the cannula.
